(19) United States
(12) Patent Application Publication
(10) Pub. No.: US 2006/0240124 A1
(43) Pub. Date: Oct. 26, 2006

(54) Treatment of anemia

(76) Inventor: Yash P. Sharma, Chantilly, VA (US)

Correspondence Address: BAKER & MCKENZIE LLP, Pennzoil Place, South Tower, 711 Louisiana, Suite 3400, HOUSTON, TX, 77002-2716, US

(21) Appl. No.: 11/113,155

(22) Filed: Apr. 25, 2005

(51) Int. Cl. A61K 31/714; A61K 31/739; A61K 31/573; A61K 31/525; A61K 33/26

(52) U.S. Cl. 424/646; 514/52; 514/54; 514/251; 514/171

(57) ABSTRACT

Methods for treating anemia in a subject are described that include administering N-glycolylneuraminic acid or a derivative thereof to the subject.

### BACKGROUND OF THE INVENTION

The condition of anemia is characterized by a lower than normal number of red blood cells (erythrocytes) in the blood, usually measured as a decrease in the amount of hemoglobin and red blood cells. Hemoglobin is a hemoprotein composed of globin and heme (an organic molecule with an iron atom) that gives red blood cells their characteristic color, and that functions primarily to transport oxygen from the lungs to the body tissues. Anemia occurs for different reasons, and these include: increased destruction of red blood cells, increased blood loss from the body, and inadequate production of red blood cells by the bone marrow, among others. In some cases anemia results from an inherited disorder, whereas in certain other cases the condition is caused by something in a person's environment, such as a nutritional problem, infection, or exposure to a drug or toxin. Common risk factors for anemia include: heavy periods, pregnancy, older age, and diseases that cause anemia, among others.

According to the National Anemia Action Council, approximately 3.4 million Americans are anemic and millions more may be undiagnosed or at increased risk of developing anemia. As many as 61% of elderly people have anemia, depending on age, sex, and overall health, and the major causes in the elderly are chronic disease and iron deficiency. Both hemoglobin (amount of hemoglobin in a set volume of blood) and hematocrit (percentage of red blood cells in a blood sample) values are used to define anemia. The normal range of hemoglobin values is 14 g/dL to 17.4 g/dL for adult men and 12.3 g/dL to 15.3 g/dL for nonpregnant women. The World Health Organization defines anemia as less than 12 g/dL for nonpregnant women and less than 13 g/dL for men. The normal average hematocrit for adult males is 46%, and the range is 40% to 52%. For adult females, the normal average hematocrit is 41% and the range is 35% to 47%. Values that fall below the lower limits can indicate anemia.

The most common symptoms of anemia are fatigue, chest pain, shortness of breath, pale complexion, and rapid heart rate. Other symptoms of various types of anemia can include: loss of appetite, diarrhea, numbness and tingling of hands and feet, joint pain, delayed growth, susceptibility to infections, ulcers on the lower legs, fever, sore mouth and tongue, brittle nails, unusual food cravings, blue tinge to sclerae, irritability, jaundice, easy bruising, nose bleed, bleeding gums, prolonged bleeding, frequent or severe infections, dark urine, enlarged spleen, and confusion or change in mental status, among others. In cases of severe anemia, it can cause low oxygen levels in vital organs, eventually leading to organ failure (i.e., heart attack).

There are important relationships between anemia and cancer, HIV/AIDS, hepatitis C, critical illness, diabetes, inflammatory bowel disease, aging, kidney disease, and surgery, among others. Chemotherapy and radiation therapy for cancer can damage the bone marrow, and cause anemia. Certain types of cancer themselves can also contribute to anemia. Kidney disease is a common complication of diabetes. The kidneys produce a hormone called erythropoietin, which stimulates bone marrow to produce red blood cells. Iron is also needed for red blood cell production. Damaged kidneys do not produce enough erythropoietin, and as a result anemia can develop.

As a result of the inflammation associated with HIV/AIDS a patient may not be able to produce enough red blood cells. Less common causes for HIV-associated anemia include vitamin B₁₂ deficiency, and the autoimmune destruction of red blood cells. Some of the early drugs used to treat HIV/AIDS, such as AZT, were shown to be a possible cause of anemia. However, the newer highly active anti-retroviral therapy (HAART) drugs are much less likely to cause anemia.

Certain chronic diseases (i.e., hepatitis C) can interfere with the production of red blood cells, resulting in chronic anemia. In anemia of chronic disease a body can not use its stored iron. Erythropoietin is suppressed and the bone marrow does not respond normally. Red blood cells may also have a shortened life span. Chronic diseases that can lead to this type of anemia include: a chronic bacterial endocarditis, hepatitis C, osteomyelitis, rheumatoid arthritis, rheumatic fever, Crohn's disease, AIDS, hypothyroidism, leukemia, cirrhosis, enlarged spleen, and ulcerative colitis, among others.

As many as 77% of critically ill (e.g., acute illness) patients may be anemic. Many factors can contribute to anemia in critically ill people (i.e., patients with congestive heart failure), including blood loss, infection, inflammation, and nutritional deficiencies. Anemia may have a serious effect on a patient who is critically ill, because of underlying illness or sudden blood loss from an injury or surgery.

At least 10% of people with inflammatory bowel disease (IBD) have anemia. Causes for anemia in people with IBD include blood loss from the intestines or problems taking in and absorbing enough nutrients. Another cause of anemia associated with IBD is the inflammation associated with IBD.

Anemia may be present before surgery, especially in people who have cancer or other chronic diseases. A major cause of the anemia can be the inflammation associated with these conditions. Another cause of pre-operative anemia is low levels of iron due to internal bleeding.

Many medications increase the risk for anemia. Among these are certain antibiotics, some antiseizure medications (e.g., phenytoin), immunosuppressive drugs (e.g., methotrexate and azathioprine), antiarrhythmic agents (e.g., procainamide and quinidine), anticlotting drugs (e.g., aspirin, warfarin, and heparin), and cancer treatments (including drugs and radiation), among others.

Certain types of anemia are caused by deficiency of certain factors in the body, and these include: B₁₂ deficiency, folate deficiency, iron deficiency, and glucose-6-phosphate dehydrogenase (G6PD) deficiency. Folate deficiency and B₁₂ deficiency anemia can be a form of megaloblastic anemia. Other types of anemia include anemia of chronic disease, hemolytic anemia (i.e., idiopathic autoimmune hemolytic anemia, immune hemolytic anemia, and sickle cell anemia), idiopathic aplastic anemia, pernicious anemia, and secondary aplastic anemia.

B₁₂ deficiency anemia is caused by a lack of sufficient amount of vitamin B₁₂ in the body. Vitamin B₁₂ is essential for normal nervous system function and blood cell production. Depending on the severity of the anemia, and on the cause of the deficit in vitamin B₁₂, the condition may be treated with vitamin B₁₂ injections or changes in diet combined with vitamin B₁₂ supplements. However symptoms related to the central nervous system may be irreversible if treatment is not begun within 6 months of when symptoms appear.

Pernicious anemia is a type of megaloblastic anemia, and it is caused by a lack of intrinsic factor, a protein needed to absorb vitamin B₁₂ from the gastrointestinal tract. Causes for low levels of intrinsic factor include: atrophic gastric mucosa, autoimmunity against gastric parietal cells, and autoimmunity against intrinsic factor, among others. Very rarely, infants and children are found to have been born lacking the ability to produce effective intrinsic factor. Although a juvenile form of the disease can occur in children, pernicious anemia usually does not appear before the age of 30. B₁₂ can be injected, introduced orally, or introduced intranasally to treat pernicious anemia.

Folate is necessary for red blood cell formation and growth, and it is usually obtained by consumption of green, leafy vegetables and/or liver. A continual dietary supply of folate is needed for normal red blood cell formation and growth. Poor dietary intake of folate, chronic alcoholism, malabsorption diseases, certain medications, and pregnancy can all be contributing factors to this type of anemia. Treatment of this type of anemia can involve oral or intravenous folic acid supplements, or modifying a patient's diet.

Megaloblastic anemia is a blood disorder characterized by red blood cells that are larger than normal. Deficiencies of vitamin B₁₂ and folic acid are the most common causes of megaloblastic anemia. Other causes are leukemia, myelofibrosis, multiple myeloma, certain hereditary disorders, drugs that affect DNA synthesis such as chemotherapy agents (methotrexate), alcohol, and other causes. The treatment for megaloblastic anemia depends on the cause of the anemia.

Iron deficiency anemia is most common form of anemia. Approximately 20% of women, 50% of pregnant women, and 3% of men are iron deficient. Iron is an essential component of hemoglobin. Iron is normally obtained through dietary intake and by recycling of red blood cells. Iron deficiency can result from a dietary deficit, poor absorption by the body, or loss of blood. It can also be related to lead poisoning in children. Iron deficiency anemia can also be caused by gastrointestinal blood loss associated with ulcers, the use of aspirin or nonsteroidal anti-inflammatory medications, or certain types of cancer (i.e., cancer of the esophagus, stomach, and colon, among others). Iron deficiency anemia can be treated with oral, intravenous, or intra-muscular iron. Modifying the patient's diet can also improve iron deficiency anemia, in some cases.

Hemolytic anemia is caused by premature destruction of red blood cells. It occurs when the bone marrow is unable to compensate for premature destruction of red blood cells by increasing their production. Causes of hemolytic anemia include infection, certain medications, autoimmune disorders, and inherited disorders, among others. Types of hemolytic anemia include: sickle-cell anemia, paroxysmal nocturnal hemoglobinuria, hemoglobin SC disease, anemia due to a G6PD deficiency, hereditary elliptocytosis, hereditary spherocytosis, hereditary ovalocytosis, idiopathic autoimmune hemolytic anemia, non-immune hemolytic anemia caused by chemical or physical agents, secondary immune hemolytic anemia, and thalassemia. Severe anemia can aggravate certain conditions, such as pre-existing heart disease, lung disease, or cerebrovascular disease, among others. Treatment depends upon the type and cause of the hemolytic anemia. Folic acid, iron replacement, and corticosteroids may be used in treatment of hemolytic anemia. In emergencies, transfusion of blood may be necessary.

Idiopathic autoimmune hemolytic anemia results from an abnormality of the immune system that destroys red blood cells prematurely. The cause is unknown. Autoimmune hemolytic anemia can be treated with certain corticosteroids (i.e., prednisone) and/or splenectomy. Immunosuppressive therapy is given if the person does not respond to corticosteroids and splenectomy. Imuran and Cytoxan have both been used. Blood transfusions are given with extreme caution, as they may result in harmful reactions.

Immune hemolytic anemia is characterized by premature destruction of red blood cells by the immune system. It results when antibodies form against the body's own red blood cells. The antibodies may be acquired by blood transfusion, pregnancy (if the baby's blood type is different from the mother's), as a complication of another disease, or from a reaction to medications. If the cause of antibody formation is disease or medication, it is referred to as a secondary immune hemolytic anemia. The antibodies will destroy the blood cells because they are recognized as foreign substances within the body. The cause may often be unknown.

Hemolytic anemia caused by a G6PD deficiency is due to a hereditary, sex-linked enzyme defect that results in the breakdown of red blood cells when the person is exposed to stress of infection or certain drugs. People with the disorder are not normally anemic and display no evidence of the disease until the red blood cells are exposed to an oxidant or stress, and then they can develop acute hemolytic or a chronic spherocytic type anemia. Drugs that can bring on the anemia include: antimalarial agents, sulfonamides, aspirin, nonsteroidal anti-inflammatory drugs, nitrofurantoin, quinidine, and quinine, among others.

Idiopathic aplastic anemia is a failure of the bone marrow to properly form all types of blood cells (red and white blood cells and platelets). It is a condition that results from injury to the stem cell, a cell that gives rise to other cell types after it divides. The cause of idiopathic aplastic anemia is not known, but is thought to be an autoimmune process. Other types of aplastic anemia may be due to chemotherapy, radiation therapy, toxins, drugs, pregnancy, congenital disorder, or systemic lupus erythematosus, among others. Aplastic anemia can be treated with blood and platelet transfusions. Severe aplastic anemia is a life-threatening condition, and bone marrow transplants may be indicated for severe disease in younger patients. Antithymocyte globulin (ATG), a horse serum that contains antibodies to human T cells, may be used as an alternative treatment. ATG is used to attempt to suppress the body's immune system, allowing the bone marrow to resume its blood cell generating function. Other medications to suppress the immune system may be used, such as cyclosporine and cyclophosphamide, among others. Corticosteroids and androgens have also been used for some cases of aplastic anemia untreated aplastic anemia is an illness that leads to rapid death.

Sickle cell anemia is an inherited disease in which the red blood cells, normally disc-shaped, become crescent shaped. As a result, they function abnormally and cause small blood clots. These clots give rise to recurrent painful episodes called “sickle cell pain crises.” Sickle cell anemia may become life-threatening, when damaged red blood cells break down, when the spleen enlarges and traps the blood cells, or when a certain type of infection causes the bone marrow to stop producing red blood cells. Repeated crises can cause damage to the kidneys, lungs, bones, eyes, and central nervous system. Sickle cell patients often require folic acid supplementation. Unfortunately, treatment of sickle cell disease generally has to focus on its symptoms, rather than on the disease itself. Treatment with hydroxyurea has been helpful to some patients, but there has been some concern that it may cause leukemia. Newer drugs are being developed, and some work by trying to induce the body to produce more fetal hemoglobin or by increasing the binding of oxygen to sickle cells. Bone marrow transplants are currently the only potential cure for sickle cell anemia.

Clearly the symptoms of anemia can be unpleasant and, in some cases, life-threatening. New methods of treating anemia are desirable.

N-glycolylneuraminic acid (Neu5Gc) is a cell surface sialic acid. Sialic acids, such as Neu5Gc, are N-acyl derivatives of neuraminic acid. Sialic acids occur in many polysaccharides, glycoproteins, and glycolipids in both animals and bacteria. Neu5Gc is widespread throughout the animal kingdom. However, due to a genetic mutation in the human gene that is expressed as acetyl neuraminic acid hydroxylase (which hydrolyzes acetyl neuraminic acids), Neu5Gc is nearly absent on the cell surface of human cells.

### SUMMARY OF THE INVENTION

Certain aspects of the present invention are directed to methods for treating a patient having an anemia. The methods involve administering a composition to the patient. The composition contains a therapeutically effective amount of at least one of a N-glycolylneuraminic acid or a derivative thereof, and at least one pharmaceutically acceptable excipient. In certain embodiments, the composition can also contain at least one of vitamin B₁₂, iron, folic acid, a corticosteroid, an androgen, or a blood product. The composition can have a catalytic amount of at least one pharmaceutically acceptable acid or a salt thereof, in certain embodiments. In some embodiments, the composition can be in the form of a tablet, a lozenge, a sucker, a semi-soft candy, a gum, a gel, a paste, a spray, a mouthwash, or a film. In some embodiments, the patient has a chronic disease, such as HIV infection/AIDS, that is linked to his anemia. In certain aspects of the present invention, the patient is not anemic after administration of the composition.

Certain embodiments of the present invention are directed to therapeutic compositions having at least one of a N-glycolylneuraminic acid or a derivative thereof, at least one pharmaceutically acceptable excipient, and at least one of vitamin B₁₂, iron, folic acid, a corticosteroid, an androgen, or a blood product.

Other features and advantages of the invention will be apparent from the following detailed description, and from the claims.

### BRIEF DESCRIPTION OF THE DRAWINGS

FIG. 1 is a chromatograph that depicts the HPLC Profile of Active Component of Cell Lysates (C18 column; 0.1% TFA/water and a 0-100% ACN gradient; UV detection A280).

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

“Anemia” is characterized by a lower than normal number of red blood cells (erythrocytes) in the blood. The normal range of hemoglobin values is 14 g/dL to 17.4 g/dL for adult men and 12.3 g/dL to 15.3 g/dL for nonpregnant women. Anemia refers to a hemoglobin value of less than 12 g/dL for nonpregnant women and less than 13 g/dL for men. The normal hemoglobin value of newborns falls between about 14 and 20 g/dL. At one week the range is between about 15 and 20 g/dL, and one month between about 11 and 15 g/dL. For children the normal range is between about 11 and 13 g/dL. In men after middle age the normal range is between about 12.4 and 14.9 g/dL, and for women after middle age between about 11.7 and 13.8 g/dL.

The normal average hematocrit for adult males is 46%, and the range is 40% to 52%. For adult females, the normal average hematocrit is 41% and the range is 35% to 47%. The normal range for hematocrit for newborns is between about 50 and 62%. Newborns typically have a hematocrit value of between about 55 and 68%. The normal range for a child one week of age is between about 55 and 68%, for a one month old child between about 37 and 49%, and for a child three months of age between about 30 and 36%. At one year a normal range for a child is between about 29 and 41%, and at ten years old the normal range is between about 36 and 40%. Values that fall below the lower limits may indicate anemia.

In methods of the present invention, the term “therapeutically effective amount” means an amount of a treatment component of the method that results in a meaningful patient benefit. Such patient benefits may include an increase in hemoglobin and/or hematocrit values of an anemic patient. In some cases, a therapeutically effective amount may result in hemoglobin and/or hematocrit values of an anemic patient returning to within what is considered the normal range.

“Vitamin B₁₂” refers to cobalamin, which is used in formation of red blood cells, and in maintaining a healthy nervous system. Vitamin B₁₂ contains cobalt, and is found primarily in meat, eggs and dairy products. B₁₂ is necessary for rapid synthesis of DNA during cell division. This is especially important in tissues where cells are dividing rapidly, particularly the bone marrow tissues responsible for red blood cell formation.

“Folic acid” is a collective term for pteroylglutamic acids and their oligoglutamic acid conjugates. N-[p-[[(2-Amino-4-hydroxypteridin-6- yl)methyl]amino]benzoyl]-L(+)-glutamic acid, specifically, pteroylmonoglutamic acid, is the growth factor for Lactobacillus casei, and is a member of the vitamin B complex necessary for the normal production of red blood cells. It is present, with or without L(+)-glutamic acid moieties, in peptide linkages in liver, green vegetables, and yeast. Folate is the term for the anion form of folic acid.

“Blood products” refers to whole blood, red blood cell components of whole blood, and platelets used in transfusions for treatment of certain anemic patients. The red blood cell components may be red blood cells, washed red blood cells, leukoreduced red blood cells, and pediatric/divided RBC units.

As used herein, “buccal administration” refers to oral administration of a composition to a patient that is held in the mouth and is used to deliver N-glycolylneuraminic acid or a derivative thereof into a patient's body. Certain compositions of the present invention may, for example, be held in the patient's mouth and sucked, to release N-glycolylneuraminic acid or a derivative thereof into the buccal cavity.

Regarding “N-glycolylneuraminic acid or derivatives thereof,” N-glycolylneuraminic acid (C₁₁H₁₉NO₁₀, MW 325.3) is the hydroxylated derivative of N-acetylneuraminic acid (sialic acid). As used herein, “derivative” refers to a compound that is similar in structure to N-glycolylneuraminic acid (Neu5Gc), such as the compounds described in U.S. Pat. Nos. 4,774,326 and 4,774,327, and N-linked glycans, as well as compounds such as glucosamine 1-acetylneuraminic acid and mannosamine acetylneuraminic acid. Additional non-limiting examples of derivatives include phosphorylated or sulfated N-glycolylneuraminic acid, N-glycolylneuraminic acid salts, O-glycolylneuraminic acid, as well as other substituted N-glycolylneuraminic acid compounds. Neu5Gc and its derivatives may be produced through chemical synthesis. As an alternative to chemical synthesis, Neu5Gc and its derivatives may be recovered from biological samples, especially animal tissues. It may be more cost effective to use unpurified or partially purified biological extracts. Neu5Gc and its derivatives may also be produced using a genetic expression system.

“Bound N-glycolylneuraminic acid or derivatives thereof” refers to N-glycolylneuraminic acid (Neu5Gc) or derivatives thereof that interact chemically with glycolipids, glycoproteins, other glycoconjugates, or phospholipids. The Neu5Gc or derivatives thereof present in certain non-human animals are often found bound to glycoproteins, glycolipids, or other glycoconjugates, or phospholipids. However, in order for Neu5Gc or derivatives thereof to be more effective in producing an immune response in humans, it may be preferable that they be introduced into the body in a chemically free form. Bound Neu5Gc or derivatives thereof may be isolated from phospholipids or glycoconjugates using nontoxic agents to disrupt their chemical interaction with these compounds.

As used herein, “pharmaceutically acceptable” refers to substances that are generally regarded as safe for introduction into the human body.

An “excipient” refers to an inert substance used in compositions of the present invention to make them easier to administer.

A “therapeutic agent” refers to a compound that is used to treat a specific disease or a medical condition.

“Essential oil” refers to a natural oil with a distinctive scent secreted by the glands of certain aromatic plants having terpenes as the major component. Examples of essential oils include, but are not limited to, citrus oils, flower oils (e.g., rose and jasmine), and oil of cloves.

“Nonnutritive sweetener” refers to a synthetic or natural substance whose sweetness is higher than or comparable to sucrose, and which may have properties such as reduced cariogenicity, health benefits for diabetics, or reduced caloric value compared to sugars.

“Catalytic amount” refers to having a sufficient amount of an acid or salt thereof in a composition of the present invention, such that it may act as a catalyst for freeing at least some N-glycolylneuramic acid or a derivative thereof from interactions with glycoconjugates or phospholipids.

Certain embodiments of the present invention are directed to methods for treating a patient having anemia. Methods of the present invention involve administering to a patient having anemia a composition. The composition contains a therapeutically effective amount of at least one of a N-glycolylneuraminic acid or a derivative thereof, and at least one pharmaceutically acceptable excipient. The patient may have anemia that is a B₁₂ deficiency anemia, a folate deficiency anemia, an iron deficiency anemia, a glucose-6-phosphate dehydrogenase deficiency, a hemolytic anemia, an aplastic anemia, or a pernicious anemia, in some embodiments. In some aspects of the present invention the patient may have a B₁₂ deficiency anemia, a folate deficiency anemia, or an iron deficiency anemia. In some embodiments, the patient may have an anemia of chronic disease. Anemia of chronic disease may be associated with chronic bacterial endocarditis, hepatitis C, osteomyelitis, rheumatoid arthritis, rheumatic fever, Crohn's disease, hypothyroidism, leukemia, cirrhosis, enlarged spleen and ulcerative colitis, among others. In certain embodiments, the patient may have anemia associated with HIV infection and/or AIDS.

After the administration of an effective amount of a composition of the present invention, in some instances, the anemic patient's hemoglobin and hematocrit levels may increase. In some cases, the anemic patient may no longer be anemic after administration of an effective amount of the composition. That is, an initially anemic adult non-pregnant female patient, may have a hemoglobin level of between about 12 g/dL and about 15.3 g/dL after administration, in certain embodiments. In some embodiments an initially anemic adult male, may have a hemoglobin level of between about 13 g/dL and about 17.4 g/dL after administration of a composition of the present invention. After administration of a composition of the present invention, an adult non-pregnant female may have a hematocrit between about 35% and about 47%, and an adult male may have a hematocrit between about 40% and about 52%, in some embodiments.

In certain embodiments, the composition may contain at least one of vitamin B₁₂, iron, folic acid, a corticosteroid, an androgen, or a blood product. The composition may have at least one of vitamin B₁₂, iron, or folic acid, in some embodiments. In certain embodiments the composition may contain whole blood, red blood cells, or platelets.

In some embodiments, the composition may have between about 0.002 wt % and 20 wt % of the N-glycolylneuraminic acid or the derivative thereof. In other embodiments, the composition may have between about 0.03 wt % and 20 wt % of the N-glycolylneuraminic acid or the derivative thereof, and in some, between about 0.03 wt % and 10 wt % of the N-glycolylneuraminic acid or the derivative thereof. In certain embodiments, the composition may have a derivative of N-glycolylneuraminic acid, and the derivative may be a phosphorylated N-glycolylneuraminic acid or a sulfated N-glycolylneuraminic acid, among others known in the art. In certain aspects of the present invention, the composition may have synthetic N-glycolylneuraminic acid or a derivative thereof. Synthetic Neu5Gc is available from Sigma Aldrich, St. Louis, Mo. In some embodiments, the composition may have Neu5Gc or a derivative thereof that is a product of a genetic expression system.

The composition may have bound or free N-glycolylneuraminic acid or a derivative thereof that has been extracted from a biological sample in some aspects of the invention. In certain embodiments, the composition may include a biological sample that contains the N-glycolylneuraminic acid or a derivative thereof. In particular, N-glycolylneuraminic acid may be obtained from or be a component of at least one of a sea cucumber extract; a peripheral blood mononuclear cell extract of a non-human animal (e.g., a pig or baboon PBMC extract); a submaxillary gland extract of a non-human animal (e.g., a equine, bovine or porcine submaxillary gland extract); an extract from meat or meat fat (e.g., beef, pork, lamb, and poultry) consumed by humans; a milk, butter or cheese extract (e.g., bovine, goat, and sheep milks and cheeses); a fish extract (e.g., cod, tuna, and salmon extracts), a starfish extract, a shark extract, a crocodile extract, or a sea urchin extract, among others known in the art. In certain aspects of the present invention, a sea cucumber extract, a pig submaxillary gland extract, a salmon extract, a milk, butter, or cheese extract, a lamb extract, a pork extract, a beef extract, or a beef fat extract may be used as the source of bound or free N-glycolylneuraminic acid or derivatives thereof in the composition. In some embodiments of the present invention, a sea cucumber extract, a meat extract, a meat fat extract, or a goat cheese extract may be used. In some aspects of the present invention, a sea cucumber extract may be used. Extracts comprising N-glycolylneuraminic acid or derivatives thereof may be purchased commercially from, for example, Sigma Aldrich as a porcine submaxillary gland extract. (PNAS 100, October, 2003)

In some embodiments, the composition may be administered to a patient having an anemia may have at least some N-glycolylneuraminic acid or derivative thereof that is bound, and the composition may further have a catalytic amount of at least one pharmaceutically acceptable acid or a salt thereof. Certain compositions of the present invention may have between about 0.01 wt % and 50 wt % of an acid or a salt thereof. In certain aspects of the present invention, the compositions may have between about 0.01 wt % and 5 wt % of an acid or a salt thereof. In some embodiments, the compositions may have between about 0.1 wt % and 5 wt % of an acid or a salt thereof. Examples of acids that may be used in the present invention include: salicylic acid, glycolic acid, phosphoric acid, pentathoic acid, and ascorbic acid, among others known in the art. The composition may contain salts of such acids.

In certain aspects, the composition may have ascorbic acid or salt thereof. Examples of ascorbic acid salts that could be used in compositions of the present invention include mono-, di-, and tri-sodium citrate salts of ascorbic acid, among others. Such acids or salts thereof are available commercially from Sigma Aldrich, St. Louis, Mo. Certain compositions of the present invention may contain between about 0.002 wt % and 20 wt % ascorbic acid or a salt thereof. In some embodiments, the compositions contain between about 0.01 wt % and 5 wt % ascorbic acid or a salt thereof. In certain embodiments, the compositions contain between about 0.1 wt % and 5 wt % ascorbic acid or a salt thereof.

In some embodiments, the composition may be administered intravenously, parenterally, intranasally, transdermally, or buccally. In certain embodiments, the composition may be administered, intravenously, parenterally or buccally. The composition may be administered buccally in some embodiments of the present invention. The composition may be in the form of a tablet, a lozenge, a sucker, a semi-soft candy, a gum, a gel, a paste, a spray, a mouthwash, or a film, in certain embodiments of the present invention. The at least one pharmaceutical excipient in compositions used in methods of the present invention may be those known in the art that are appropriate to the method of administration. The excipient may be present in an amount greater than about 50 wt % by weight (wt %), in some embodiments in an amount greater than about 80 wt %, and in certain embodiments, in an amount greater than about 90 wt % in a composition of the present invention. Components of a tablet, a lozenge, a sucker, a semi-soft candy, a gum, a gel, a paste, a spray, a mouthwash, or a film of the present invention preferably do not interfere with the therapeutic properties of N-glycolylneuraminic acid or derivative thereof.

The composition used in methods of the present invention may be administered buccally, in some embodiments. When administered buccally, the composition may be ingested allowing systemic introduction of N-glycolylneuraminic acid or derivative thereof, or the composition may be applied to a surface of the oral cavity (e.g., gums, teeth, cheeks, under the tongue, etc.) or a surface of a dental prosthesis. When used buccally, the composition may be in the form of a tablet, a lozenge, a sucker, a semi-soft candy, a gum, a spray, a gel, a film, a paste, or a mouthwash, among other forms known in the art.

The composition in the form of a gel or a paste may be applied to a surface of the oral cavity (i.e., gums), in certain embodiments. In some embodiments, the gel or paste may be a toothpaste or gel used to brush the teeth. Compositions used in the methods of the present invention may have components of pastes or gels known in the art that do not interfere with the action of N-glycolylneuraminic acid or derivatives thereof. In some embodiments, the composition in the form of a gel or a paste may contain a thickener, or a flavoring, among other components known in the art.

When a composition of the present invention is in the form of a film that is used buccally, the film may be soluble, so that it dissolves in the mouth. In some embodiments, the film may be laid on the tongue or it may be applied to other surfaces of the oral cavity. The composition may contain components of soluble film known in the art, in certain embodiments. In some embodiments, the film may be insoluble, and the composition may be impregnated in the film or applied to its surface. The composition may be diffuse from the insoluble film into the oral cavity. The film may be applied to the surface of the teeth, or between the gums and cheek, and the film may be removed after diffusion occurs. Such insoluble films may, in some embodiments, contain tooth whiteners that do not prevent absorption of the N-glycolylneuraminic acid or a derivative thereof.

When a composition of the present invention is in the form of a spray, it may be used buccally or intranasally. When used intranasally, the spray may consist essentially of a mist of liquid or a puff of a fine powder. A spray used in methods of the present invention, may contain elements known in the art for use in sprays. In certain embodiments, a spray may have at least one of a propellant, among others components known in the art. In certain embodiments, a composition of the present invention may be swabbed in the inside of the nose. Components of the composition used to swab the nose may include those known in the art that will permit the Neu5Gc or a derivative thereof to have a therapeutic effect.

Examples of excipients that could be included with the composition when administered buccally include: mannitol, cyclodextrins and their derivatives magnesium stearate, calcium carbonate, sodium carbonate, lactose, D-mannitol, calcium phosphate, sucrose, sodium chloride, glucose, starch, kaolin, cellulosic materials, anhydrous calcium secondary phosphate, light anhydrous silicic acid, partly pregelatinized starch, acacia powder, gum arabic, sorbitol, corn starch, wax, or alginic acid, among others.

According to some embodiments, the excipient in compositions of the present invention may serve more than one role in the composition. For example, mannitol may function as both a nonnutritive sweetener and an excipient. Similarly, the excipient may serve as a flavorant, buffering agent, lubricant, or other component of the composition.

In certain embodiments involving buccal administration, a composition of the present invention may be at least partially dissolved by saliva in the patient's mouth. In some embodiments, involving buccal administration, at least some of the N-glycolylneuraminic acid or derivative thereof may be bound, and the composition may have a catalytic amount of at least one pharmaceutically acceptable acid or a salt thereof, and at least some of the acid or salt thereof may be dissolved by saliva in the patient's mouth. In certain embodiments, compositions for buccal administration of the present invention may have at least one extract having at least one bound N-glycolylneuraminic acid or a derivative thereof, at least one of ascorbic acid or a salt thereof, and at least one pharmaceutically acceptable excipient. In certain embodiments, the composition may have between about 0.002 wt % and 20 wt % bound N-glycolylneuraminic acid or a derivative thereof, and between about 0.02 wt % and 50 wt % ascorbic acid or a salt thereof.

In some embodiments, compositions of the present invention may further have at least one pharmaceutically acceptable component selected from a coloring agent, a polypeptide, a coating, a sweetener, a flavoring, an antibacterial agent, a taste modifier, a preservative, a disintegrator, a disintegration-preventor, a binder, an antioxidant, a dietary supplement, an antiblocking agent, an antisticking agent, an absorption promoter, absorption-adsorption carriers or a therapeutic agent, a paste thickener, a propellant or a therapeutic agent, among others known in art. Therapeutic agents may, in some embodiments, be a pain reliever. Such components when present in certain embodiments do not interfere with release and absorption of N-glycolylneuraminic acid. As with the excipient, some of these components of a composition may serve in more than one role.

Examples of disintegrators, include dry starch, alginic acid, agar powder, crosslinked polyvinyl pyrrolidone, crosslinked sodium carboxymethylcellulose, L-hydroxypropylcellulose, calcium carboxymethylcellulose, and sodium starch glycolate, among others. Examples of disintegration-preventors, are stearyl alcohol, stearic acid, cacao butter, and hydrogenated oil, among others. Binders such as gelatin, crystalline cellulose, simple syrup, sucrose, glucose solution, starch solution, polyvinyl alcohol, polyvinyl ether, polyvinylpyrrolidone, carboxymethylcellulose, shellac, methylcellulose, ethylcellulose, sodium alginate, gum arabic, hydroxypropylmethylcellulose, hydroxypropylcellulose, water, D-mannitol, dextrin, ethanol, starch, gelatin, and acacia, among others may be used in certain compositions.

Antiblocking and antisticking agents such as aluminum silicate, calcium hydrogen phosphate, magnesium oxide, talc, and silicic acid anhydride, among others, may be used in certain compositions of the present invention. Lubricants such as magnesium stearate, calcium stearate, stearic acid, carnauba wax, light silicic acid anhydride, aluminum silicate, magnesium silicate, hardened oil, hardened vegetable oil derivatives, colloidal silica, sesame oil, bleached bees wax, titanium oxide, dry aluminum hydroxide gel, calcium hydrogen phosphate, sodium lauryl sulfate, polyethylene glycol, and talc, among others, may be used in certain compositions of the present invention. In certain embodiments, the lubricant may be present in an amount between about 0.1 and 25 wt %, in certain embodiments in an amount between about 0.1 and 10 wt %, and in certain aspects of the invention in an amount between about 0.1 and 5 wt % of the inventive composition. Examples of absorption promoters that may be used in certain embodiments of the present invention include quaternary ammonium salts, sodium lauryl sulfate, urea, and enzymes, among others. Examples of absorption-adsorption carriers are starch, lactose, kaolin, bentonite, silicic acid anhydride, hydrated silicon dioxide, magnesium metasilicate-aluminate, and colloidal silicic acid, among others.

Further, if desired, a tablet, a semi-soft candy, a gum, a sucker, or a lozenge may be coated. The coating may be made with sugar, or gelatin, among others compounds. The coating may have hydroxypropylmethylcellulose, ethylcellulose, hydroxymethylcellulose, hydroxypropylcellulose, polyoxyethylene glycol, Tween 80, Pluronic F68, cellulose acetate phthalate, hydroxypropylmethylcellulose phthalate, hydroxymethylcellulose acetate succinate, Eudragit (methacrylic acid/acrylic acid copolymer, manufactured by Rohm and Haas, Del.), or pigment (e.g., iron oxide red, titanium dioxide, et.), among other coating components known in the art.

Flavorings that may be used in the present invention include sweeteners, especially non-nutritive sweeteners. Examples of flavorings that could be used in certain embodiments, include acacia or tragacanth, among others. Certain compositions of the present invention may contain more than one sweetener. In certain embodiments, the flavoring has a nonnutritive sweetener that is noncariogenic. The cariogenicity of a substance is dependent upon its susceptibility to fermentation by Streptococcus mutans and other oral microorganisms. Dental researchers have long recognized that fermentable sweeteners such as sucrose, glucose, starch, and corn syrup are cariogenic or cavity causing. Examples of nonnutritive sweeteners that may be used in compositions of the present invention include: saccharin, invert sugar, cyclamate, palantinose, aspartame, xylitol, acesulfame, sorbitol, monellin, mannitol, meohesperidine, maltitol, and palatinit, among others. In certain compositions of the present invention, the nonnutritive sweetener may be present in an amount between about 50 and 90 wt %, in certain embodiments in an amount between about 70 and 90 wt %, and in some embodiments in an amount between about 80 and 90 wt %.

Other flavorings that may be used in compositions of the present invention include a candy taste, such as chocolate, orange, vanilla, and the like; essential oils such as peppermint, spearmint and the like; or other flavor, such as anis seed, eucalyptus, 1-menthol, carvone, and anethole, among others known in the art. Both individual and mixed flavors are contemplated. The flavorings are generally utilized in amounts that will vary depending upon the individual flavor, and may, for example, range in amounts of about 0.1% to about 6% by weight of the final composition.

In certain embodiments, fluoride, and more particularly sodium monofluorophosphate or sodium fluoride may be incorporated into a composition of the present invention, especially one having a nonnutritive sweetener, such as xylitol.

The coloring agents useful in the present invention include pigments which may be incorporated in amounts of up to about 2% by weight of the composition. Also, the coloring agents may include other dyes suitable for food, drug and cosmetic applications (i.e., FD&C dyes) and the like. The materials acceptable for the foregoing spectrum of use are, in some embodiments, water-soluble. Illustrative examples include the indigo dye known as FD&C Blue No. 2, which is the disodium salt of 5,5-indigotindisulfonic acid, FD&C Green No. 1, which is a triphenylmethane dye and is the monosodium salt of 4-[4-N-ethyl-p-sulfobenzyl amino)diphenyl-methylene]-[1-(N-ethyl-N-p-sulfoniumbenzyl)-2,5-cyclohexadienimine]. Other FD&C and D&C colorants useful in the present invention and their corresponding chemical structures may be found in the Kirk-Othmer Encyclopedia of Chemical Technology, 3rd Edition, in Volume 6, at pages 561-595.

The amount of N-glycolylneuraminic acid or a derivative thereof effective for treating an anemia in a subject may vary, depending on a number of factors, including the amount of the composition in individually prepared doses (e.g., tablets) conveniently available, the chemical characteristics of the compounds employed, the formulation of the compound excipients and the route of administration. The optimal dosage of N-glycolylneuraminic acid to be administered also may depend on such variables as the overall health status of the particular patient and the relative biological efficacy of the compound (e.g., N-glycolylneuraminic acid or derivatives thereof) selected. Compositions of the present invention may have between about 0.002 wt % and 20 wt % bound or free N-glycolylneuraminic acid or a derivative thereof. In some embodiments, the compositions may have between about 0.01 wt % and 5 wt % bound or free N-glycolylneuraminic acid or a derivative thereof. In certain embodiments, the compositions may have between about 0.01 wt % and 2.5 wt % bound or free N-glycolylneuraminic acid or a derivative thereof.

Dosing regimens that may be used in the present invention include administering the composition at least once per day. In certain embodiments, the dosing regimen involves administering the composition at least three times per day. In some embodiments, the dosing regimen involves administering the composition at least four times per day. In addition, delayed release formulations of the composition may be used such that administrations are less frequent. In certain embodiments, a physician may prescribe the proper dosages and dosing regimen. The composition may be, in certain embodiments, in unit dosage form. In such form the composition is subdivided into unit doses containing appropriate quantities of the active component. The unit dosage form may be a packaged preparation, the package containing discrete quantities of preparation, such as packeted tablets or lozenges. In certain embodiments, the unit dosage form may be a tablet or lozenge itself, or it may be the appropriate number of any of these in packaged form. In some embodiments of the present invention, the composition may be administered in addition to at least one additional anemia treatment.

The dosing regimen in certain aspects of the present invention may involve administering to the patient between about 0.1 mg and 1000 mg of the composition per day. In some embodiments, between about 0.2 mg and 100 mg of the composition may be administered per day, and in certain embodiments, between about 0.2 mg and 80 mg of the composition may be administered per day.

### EXAMPLES

### Example 1

### Isolation of a Small Molecule from Baboon Blood

Peripheral blood monocytes (PBMC) were isolated from whole baboon blood using Ficoll-Hypaque density gradient centrifugation or from PBMCs further expanded in tissue culture following activation with phytohemagglutinin-P (PHA-P) and growth in medium containing interleukin-2 (IL-2). In either case, the PBMCs first were washed 3 times with sterile phosphate-buffered saline (PBS) and pelleted by centrifugation. The cell pellet then was lysed by resuspension in sterile H₂O and held for 96 hours at 4° C. Proteins and nucleic acids were precipitated from the extract and the remaining components in the extract were stabilized using 10% (v/v) calcium phosphate buffer (pH 7.4) containing 0.01% calcium chloride and 0.001% ascorbic acid. The solution was clarified by centrifugation followed by filtration through a 0.22 μm filter. This final filtrate represented a 1:50 dilution of the initial cell lysate and is hereafter referred to as the cell lysate. In some instances, the cell lysate preparation was sterilized by Cobalt radiation at 2.5 mRADs for 3 hours (Neutron Products, Inc. Gaithersburg, Md.).

A series of in vitro and in vivo toxicology studies were conducted to evalute the potential adverse effects of the cell lysate on human blood cells, blood clotting factors, and in rats administered intravenous cell lysate repeatedly over 28 days. Freshly drawn heparinized whole blood (1 ml) was diluted 1:4 in PBS (pH 7.4) and 0.1 ml of cell lysate (freshly prepared) was added per ml of diluted whole blood. Two aliquots of the blood were maintained at 4° C. for 21 and 42 days. Extract was not added to the control samples (freshly drawn blood, and blood stored for 21 and 42 days). Results are reported in Table 1.

TABLE 1
Addition of cell lysate to whole blood

| | Day O | | Day 21 | | Day 42 | |
|---|---|---|---|---|---|---|
| Para- | Un- | | Un- | | Un- | |
| meter | treated | Treated | treated | Treated | treated | Treated |
| RBC | 5.3 | 5.3 | 4.2 | 5.1 | 3.0 | 5.1 |
| Hb | 12.8 | 12.9 | 8.4 | 12.8 | 6.3 | 12.6 |
| Hct | 32.3% | 32.3% | 20.2% | 33.0% | 15.8% | 32.0% |
| Lysis | 0% | 0% | 10% | 0% | 40% | 0% |
| 45% | 0% | 0% | 92% | 0% | 58.4% | 0% |
| saline | | | | | | |

The effects of cell lysate on Factor VIII and Factor IX clotting activities were determined by adding cell lysate (1:10 dilution) to human plasma and incubating the samples for 1, 2, 5, and 23 hours at 37° C. The samples were frozen and assayed for Factor VIII and Factor IX clotting activity by a one-stage activated prothrombin time (APTT) method using 0.15 M sodium chloride as the control. Denson, Br. J. Haematol., 1973, 24(4):451-461. Samples also were incubated at room temperature and at 4° C. Factor VIII and Factor IX activities of the samples treated with cell lysate (0.076 and 0.389 units/ml respectively) were not different from saline controls (Table 2,% activity remaining).

TABLE 2
Clotting Activity in Treated Samples

| Time | | | | |
|---|---|---|---|---|
| (Hrs) | FVIII/saline | FVIII/cell lysate | FIX/Saline | FIX/cell lysate |
| 0 | 91.81 | 111.11 | 91.13 | 93.01 |
| 1 | 93.92 | 114.29 | 98.77 | 103.06 |
| 2 | 80.11 | 120.91 | 96.95 | 93.83 |
| 5 | 93.5 | 114.29 | 93.51 | 93.68 |
| 23 | 67.95 | 87.45 | 40.14 | 62.03 |

In vivo toxicology of the cell lysate was examined using Sprague-Dawley CD albino rats. Five male rats and five female rats were administered 1 ml of cell lysate per day by intravenous injection into the tail vein on 6 occasions (on Days 1, 3, 5, 7, 9 and 28). All animals were examined twice daily for mortality and signs of ill health or reaction to treatment, with a more detailed examination performed weekly. There were no treatment-related clinical signs and the animals did not develop hypersensitivity reactions by the end of the experiment on Day 28. No treatment-related effects on food consumption or weight gain were observed. Blood samples were collected under anesthesia from the orbital sinus, 24 hours following the dose at day 9, and again from the abdominal aorta at necropsy at day 28. All animals were euthanized by exsanguinations from the abdominal aorta following anesthesia. At the termination of the study on Day 28, hematological evaluations were performed on all animals. There were no mortalities, no treatment-related clinical observations, or effects on white cell parameters.

### Example 2

### Cytotoxicity of the Cell Lysate on Cultured Blood Mononuclear Cells

Cultured human blood mononuclear cells were cultured in the presence of varying concentrations of cell lysate for 7 days. Solutions containing different concentrations of cell lysate were prepared by diluting the stock solution of cell lysate (1 mg/mL) 1:4, 1:20, 1:100, and 1:500 with PBS. An equal volume of each dilution of cell lysate was added to cultured cells. Medium was changed at Day 3. The resulting cell counts at each concentration of cell lysate are listed in Table 3. A calorimetric assay was used to assess cytotoxicity. A WST-1 test kit (a tetrazolium compound that is the sodium salt of 4-[3-(4-iodophenyl)-2-(4-nitrophenyl)-2H-5-tetra olio]-1,3-benzene dislocate) was used in this assay (Roche Diagnostics, Indianapolis, Ind.).

TABLE 3
Cytotoxicity Of The Cell Lysate

| Dilution (1×) | WST-1 (%) | Cell Count (%) |
|---|---|---|
| 0 | 100 | 100 |
| 4 | 84 | 78.8 |
| 20 | 98 | 106 |
| 100 | 101 | 100 |
| 500 | 104 | 106 |

### Example 3

### Identification of Active Component of Cell Lysate

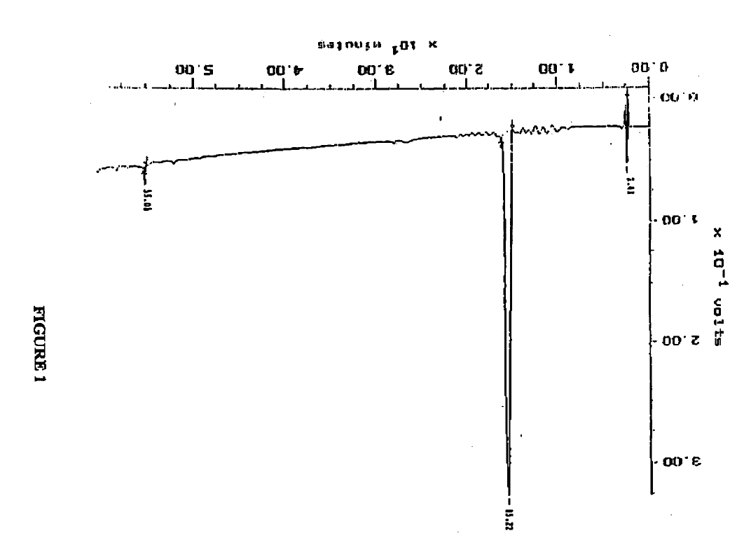

The soluble lysate isolated from PBMCs was fractionated by HULK as a first step in the identification of the active component. A C18 column (Delta Pak, 15 μm, 300_{—}, 0.39×30 cm) with a mobile phase of 0.1% tetrafluoroacetic acid (TFA) in water and a gradient of 0-100% acetonitrile (ACN) was used to separate the components in the cell lysate. One major peak eluting with 30% ACN and two minor peaks at 50% ACN were observed (FIG. 1). The 3 peaks, designated HPLC-1, HPLC-2, and HPLC-3, were collected separately and lyophilized and stored for further characterization by mass spectrometry and NMR.

Mass spectrometry was performed with a VG BioQ triple quadrupole mass spectrometer operating in the positive ion electrospray ionization mode using the following parameters: scan range m/z 100-950 and 35-700; cone voltage 57V to 63V; source temperature 80° C. to 100° C. Calibration was performed with direction injection analysis of CsI prior to LC-MS. A distinct aromatic ring absorbance with a peak maximum at 258 nm was detected. Behavior of the compound was consistent with a small molecular weight compound. Sample related masses of 86, 194, and 288 were identified. The sample related masses of 86 and 194 represented less than 1% of total. It was determined that the sample mass of 288 was composed of carbon, hydrogen, oxygen, and a single nitrogen atom.

Proton NMR also was performed on the extract in a solution of D₂O using a modified Nicolet NT 360 MH3 spectrometer operating with a single 0.5 μsec excitation pulse and a one second re-cycle delay. Signals were detected with shifts between 3.1-3.7 ppm.

The active component was identified as N-glycolylneuraminic acid based on the molecular weight and chemical composition.

### Example 4

### Assessment of Cell Proliferation in Cells Treated with N-glycolylneuraminic acid

On day 0, cell lines listed in Table 4 were plated into microtiter plates at 850-2000 cells/well in 100_L of media. On day 1, N-glycolylneuraminic acid was diluted 2× in medium and from 0 to 200 μM of N-glycolylneuraminic acid or 1000_M of N-glycolylneuraminic acid were added. The stock solution at 50/250 mM in DMSO then was diluted 1/500 in media, V_{f}=200_L/well. The cells were incubated for 3 days at 37_C and 5% CO₂. On day 4, ³[H]-thymidine, diluted 1/100 in media, was added at 25_L/well/200_L of medium, resulting in a final concentration of 0.5_Ci per well. On day 5, cells were harvested (18 hours after the addition of the ³[H]-thymidine) onto a glass fiber, and CPM/well were determined. The results are listed in Table 4.

On day 0, cell lines listed in Table 4 were plated into microtiter plates at 850-2000 cells/ well in 100 L of media. On day 1, N-glycolylneuraminic acid was diluted 2× in medium and from 0 to 200 μM of N-glycolylneuraminic acid or 1000 M of N-glycolylneuraminic acid were added. The stock solution at 50/250 mM in DMSO then was diluted 1/500 in media, V_{f}=200 L/well. The cells were incubated for 3 days at 37 C and 5% CO₂. On day 4, ³[H]-thymidine, diluted 1/100 in media, was added at 25 L/well/200 L of medium, resulting in a final concentration of 0.5 Ci per well. On day 5, cells were harvested (18 hours after the addition of the 3[H]-thymidine) onto a glass fiber, and CPM/ well were determined. The results are listed in Table 4.

TABLE 4
Treated Cell Lines

| | | Platingc | % Confluency |
|---|---|---|---|
| Cell Line | Description | pm (day 0) | (day 5) |
| Molt-4 | Peripheral blood, acute | 2000 | 50 |
| | lymphoblastic leukemia, | | |
| | human. | | |
| DU-145 | Prostate carcinoma, | 1200 | 100 |
| | metastasis to brain, | | |
| | human | | |
| HSF | Human foreskin fibroblast, | 1000 | 100 |
| | diploid, normal. | | |
| HT1080 | Fibrosarcoma, epithelial- | 850 | 100 |
| | like, human. | | |
| HepG2 | Hepatocellular carcinoma, | 2000 | 50 |
| | human. | | |

### Example 5

### Treatment of Patients Having Anemia with Lozenges Comprising N-glycolylneuraminic acid

Lozenges were manufactured by D&E pharmaceuticals, New Jersey. The lozenges contained sea cucumber extract available from Global Nutrients, New Jersey. All the lozenges were prepared from a single lot of sea cucumber extract. The lozenges contained N-glycolylneuraminic acid and/or derivatives thereof, and sodium ascorbate. The lozenges also contained magnesium stearate. Lozenges were analyzed for content by Eurofins, Petaluma, Calif.

TABLE 5

| | Total weight | N-glycolylneuraminic | Ascorbate |
|---|---|---|---|
| Sample | (mg) of tablet | acid + derivatives (mg) | (mg) |
| 1 | 500 | 0.375 | 0.22 |

Patients were administered 2 lozenges 3 times per day during treatment. The patients were instructed to hold a lozenge in the mouth until completely dissolved for 2 to 60 minutes. All patients were anemic and infected with HIV, and some were co-infected with hepatitis B and/or hepatitis C. Hemoglobin and hematocrit were measured for each patient over a period of time. The results are compiled in Table 6 below.

TABLE 6

| | | | | Hema- | | ΔHema- |
|---|---|---|---|---|---|---|
| Patient | Gender | Date | Hgb | tocrit | ΔHgb | tocrit |
| RT2 | | 7/26/2004 | 12.6 | 36.6 | | |
| | | 9/14/2004 | 12.1 | 34.7 | | |
| | | 10/1/2004 | 11.3 | 32.1 | −1.3 | −4.5 |
| TY4 | M | 8/20/2004 | 9.8 | 29.5 | | |
| | | 9/10/2004 | 10.7 | 32.8 | | |
| | | 10/8/2004 | 11.4 | 35.2 | | |
| | | 11/8/2004 | 12 | 36.1 | | |
| | | 1/7/2005 | 11 | 32.7 | 1.2 | 3.2 |
| RT19 | | 8/24/2004 | 12.2 | 33.4 | | |
| | | 9/24/2004 | 10.6 | 31.3 | | |
| | | 10/21/2004 | 11.2 | 32.4 | −1.0 | −1.0 |
| RS22 | M | 8/24/2004 | 10.4 | 30.7 | | |
| | | 9/14/2004 | 10 | 30.7 | | |
| | | 10/21/2004 | 9.7 | 29.3 | | |
| | | | 9.6 | 29.9 | | |
| | | | 8.6 | 25.9 | | |
| | | 12/3/2004 | 10.3 | 28.9 | | |
| | | | 9.7 | 27.1 | −0.7 | −3.6 |
| CB24 | F | 8/24/2004 | 12.3 | 38 | | |
| | | 9/16/2004 | 12.3 | 38.3 | | |
| | | 10/27/2004 | 13.1 | 40.8 | 0.8 | 2.8 |
| WL25 | | 8/25/2004 | 10.5 | 32.7 | | |
| | | 9/29/2004 | 10.6 | 33.7 | 0.1 | 1.0 |
| DB27 | M | 8/26/2004 | 12.5 | 37.2 | | |
| | | 9/17/2004 | 11.2 | 34.2 | | |
| | | 10/8/2004 | 11 | 34 | | |
| | | 11/5/2004 | 11.1 | 32.8 | −1.4 | −4.4 |
| MT30 | M | 8/27/2004 | 12.4 | 37.9 | | |
| | | 9/15/2004 | 12 | 36.9 | | |
| | | 10/6/2004 | 11.7 | 35.8 | | |
| | | | 12.8 | 39.3 | | |
| | | | 12.8 | 36.7 | 0.4 | −1.2 |
| DP37 | M | 10/1/2004 | 12.3 | 37.7 | | |
| | | | 11.3 | 31.9 | | |
| | | | 11.2 | 31.8 | | |
| | | | 10.9 | 31.6 | −1.4 | −6.1 |
| RV43 | M | 9/28/2004 | 10.6 | 32.7 | | |
| | | | 9.8 | 29.7 | | |
| | | | 9.4 | 28.8 | | |
| | | 11/2/2004 | 9.2 | 27.4 | | |
| | | 11/8/2004 | 9.1 | 27.4 | | |
| | | | 11 | 33 | | |
| | | | 11 | 33.8 | | |
| | | | 12.8 | 37.8 | | |
| | | 1/17/2005 | 13 | 38.1 | 2.4 | 5.4 |
| PB39 | M | 9/30/2004 | 12.9 | 37 | | |
| | | 10/7/2004 | 12.2 | 36 | | |
| | | | 12.6 | 37.7 | | |
| | | | 11 | 32.4 | −1.9 | −4.6 |
| SA3 | M | 9/24/2004 | 11.2 | 34.1 | | |
| | | 10/8/2004 | 11.1 | 33.4 | | |
| | | 11/8/2004 | 12.4 | 36.5 | | |
| | | | 14.4 | 40.7 | 3.2 | 6.6 |
| VS11 | M | | 12.7 | 39.1 | | |
| | | 11/8/2004 | 12.8 | 37.9 | | |
| | | | 12.4 | 35.2 | −0.3 | −3.9 |
| RT19 | M | 9/24/2004 | 10.6 | 31.3 | | |
| | | | 11.2 | 32.4 | | |
| | | | 12.3 | 34.9 | 1.7 | 3.6 |
| AR36 | M | 10/8/2004 | 11.6 | 35.7 | | |
| | | | 11.4 | 34.4 | | |
| | | | 11.6 | 34.9 | | |
| | | | 11.2 | 33.3 | | |
| | | | 11.4 | 32.5 | | |
| | | | 11.9 | 33.9 | | |
| | | | 13.2 | 37.2 | 1.6 | 1.5 |
| JM47 | | 11/2/2004 | 12.8 | 37.6 | | |
| | | | 14.2 | 41.8 | 1.4 | 4.2 |
| TW50 | | 9/23/2004 | 11 | 48.9 | | |
| | | | 14.7 | 43.7 | | |
| | | 12/2/2004 | 13.7 | 39.3 | | |
| | | | 14.1 | 41 | | |
| | | 1/17/2005 | 14.5 | 42.1 | 3.5 | −6.8 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a patient having an anemia comprising,
administering to said anemic patient a composition comprising,
a therapeutically effective amount of at least one of a N-glycolylneuraminic acid or a derivative thereof, and
at least one pharmaceutically acceptable excipient.

2. The method of claim 1, wherein the composition further comprises at least one of vitamin B₁₂, iron, folic acid, a corticosteroid, an androgen, or a blood product.

3. The method of claim 1, wherein the composition further comprises at least one of vitamin B₁₂, iron, or folic acid.

4. The method of claim 1, wherein the anemia is a B₁₂ deficiency anemia, a folate deficiency anemia, an iron deficiency anemia, a glucose-6-phosphate dehydrogenase deficiency, a hemolytic anemia, an aplastic anemia, or a pernicious anemia.

5. The method of claim 1, wherein the anemia is a B₁₂ deficiency anemia, a folate deficiency anemia, or an iron deficiency anemia.

6. The method of claim 1, wherein the anemia is an anemia of chronic disease.

7. The method of claim 6, wherein the anemia of chronic disease is an anemia of HIV infection or AIDS.

8. The method of claim 1, wherein the patient is not anemic after administration of the composition.

9. The method of claim 8, wherein the patient is an adult non-pregnant female and her hemoglobin is between about 12 g/dL and about 15.3 g/dL after administration.

10. The method of claim 8, wherein the patient is an adult male and his hemoglobin is between about 13 g/dL and about 17.4 g/dL after administration.

11. The method of claim 8, wherein the patient is an adult non-pregnant female and her hematocrit is between about 35% and about 47% after administration.

12. The method of claim 8, wherein the patient is an adult male and his hematocrit is between about 40% and about 52% after administration.

13. The method of claim 1, wherein at least some of the N-glycolylneuraminic acid or derivative thereof is bound, and the composition further comprises a catalytic amount of at least one pharmaceutically acceptable acid or a salt thereof.

14. The method of claim 1, wherein the composition is administered intravenously, parenterally, intranasally, transdermally, or buccally.

15. The method of claim 1, wherein the composition is administered intravenously, parenterally, or buccally.

16. The method of claim 1, wherein the composition is administered buccally.

17. The method of claim 16, wherein the composition is at least partially dissolved by saliva in the patient's mouth.

18. The method of claim 16, wherein at least some of the N-glycolylneuraminic acid or derivative thereof is bound, and the composition further comprises a catalytic amount of at least one pharmaceutically acceptable acid or a salt thereof, and at least some of the acid or salt thereof is dissolved by saliva in the patient's mouth.

19. The method of claim 1, wherein the dosing regimen comprises administering the composition at least once per day.

20. The method of claim 1, wherein the dosing regimen comprises administering to the patient between about 0.1 mg and 1000 mg of the composition per day.

21. The method of claim 1, wherein the dosing regimen comprises administering to the patient between about 0.2 mg and 100 mg of the composition per day.

22. The method of claim 1, wherein the dosing regimen comprises administering to the patient between about 0.2 mg and 80 mg of the composition per day.

23. The method of claim 1, wherein the composition is administered in addition to at least one additional anemia treatment.

24. The method of claim 1, wherein the composition comprises between about 0.002 wt % and 20 wt % of the N-glycolylneuraminic acid or the derivative thereof.

25. The method of claim 1, wherein the composition comprises between about 0.1 wt % and 20 wt % of the N-glycolylneuraminic acid or the derivative thereof.

26. The method of claim 1, wherein the composition comprises between about 0.1 wt % and 10 wt % of the N-glycolylneuraminic acid or the derivative thereof.

27. The method of claim 1, wherein the composition comprises N-glycolylneuraminic acid.

28. The method of claim 1, wherein the composition comprises a derivative of N-glycolylneuraminic acid.

29. The method of claim 1, wherein the composition comprises a phosphorylated N-glycolylneuraminic acid.

30. The method of claim 1, wherein the composition comprises a sulfated N-glycolylneuraminic acid.

31. The method of claim 1, wherein composition comprises synthetic N-glycolylneuraminic acid.

32. The method of claim 1, wherein composition comprises N-glycolylneuraminic acid extracted from a biological sample.

33. The method of claim 1, wherein the composition comprises a biological sample comprising the N-glycolylneuraminic acid or the derivative thereof.

34. The method of claim 1, wherein the composition comprises N-glycolylneuraminic acid or the derivative thereof produced using a genetic expression system.

35. The method of claim 1, wherein the composition is in the form of a tablet, a lozenge, a sucker, a semi-soft candy, a gum, a gel, a paste, a spray, a mouthwash, or a film.

36. The method of claim 1, wherein the composition further comprises at least one of a coloring agent, a polypeptide, a coating, a sweetener, a flavoring, a taste modifier, a preservative, a disintegrator, a disintegration-preventor, a binder, an antioxidant, a dietary supplement, an antiblocking agent, a paste thickener, an antisticking agent, an absorption promoter, an absorption-adsorption carrier, a propellant, or a therapeutic agent that is pharmaceutically acceptable.

37. A therapeutic composition comprising:
at least one of a N-glycolylneuraminic acid or a derivative thereof,
at least one pharmaceutically acceptable excipient, and
at least one of vitamin B₁₂, iron, folic acid, a corticosteroid, or a blood product.

38. The composition of claim 37, wherein the composition comprises at least one of vitamin B₁₂, iron, or folic acid.
